SEQUENCE LISTING (12 sequences)

SEQ ID NO: 1 - DNA, 9666 bp, Unknown
  It is a comercial plasmid named as pBIG2RHPH2
ccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattatcgatgaattcgacgttaactgatattgaaggagcattttttgggcttggctggagctagtggaggtcaacaatgaatgcctattttggtttagtcgtccaggcggtgagcacaaaatttgtgtcgtttgacaagatggttcatttaggcaactggtcagatcagccccacttgtagcagtagcggcggcgctcgaagtgtgactcttattagcagacaggaacgaggacattattatcatctgctgcttggtgcacgataacttggtgcgtttgtcaagcaaggtaagtggacgacccggtcataccttcttaagttcgcccttcctccctttatttcagattcaatctgacttacctattctacccaagcatccaaatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagttcagcgagagcctgacctattgcatctcccgccgtgcacagggtgtcacgttgcaagacctgcctgaaaccgaactgcccgctgttctccagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagagtagatgccgaccgggaaccagttaacgtctagaggtcataacgtgactcccttaattctccgctcatgatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatctggcgccggccagcgagacgagcaagattggcgtcgagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagactcgacgcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctca

SEQ ID NO: 2 - DNA, 654 bp, Mortierella formosensis
atggccatcaaggaataccagcgcgagttcattgagtttgccatcaagaacgaggtcttgaagttcggagagttcaccctcaagtccggccgtatctcgccctacttcttgaacgcgggccttttcaacactggcgcctcgctctccaagatcggaaagttctacgccgctgccgtcagcgattcgggcattgagcacgacatcatctttggccccgcctacaagggtgtccctctggcctgcaccaccgtcattgccttggccgaggccccctacaacaaggacacgccctactgcttcaaccgcaaggagaagaaggaccatggtgagggcggcacgattgttggatcagcactgaagggcaaggtcctggtcattgacgatgttatcaccgccggcaccgccatccgcgagtctgtccagatcattgaggactgcaaggcccaattggctggtgttttggtcgcggtggatcgtcaggagactggcaagaacggcgacatgtctgctatccaggaggtcgagagagactttggtgtccctgtcaaggccattgtgaccatgacccacatcatgcagtacatggaggagaagggaacctatggcgagcacttgacccagatgcgcgcttaccgggagaagtacggtgtttag

SEQ ID NO: 3 - DNA, 1393 bp, Mortierella formosensis
cgacgctgacattacacatttatccgttcgccgatatagacttaaatgggacgaggagaacttgatcatcacagaggctcagaaggattcgacaatgaaaattgatgagcccaagacgccctatgttcactacgaccacgagctggacaaagtgatcgatatgaatggtaactgaaaacatggacatccagagatcttgagcacccacgacgagatacacagaaagtcatcaggcatactgacacgtccttcacgtcacgcatttctgaattttttaatttgtctaggggaaaccttctcgttagacgggggcaagacaaagcatgcttctctagcccatggtcagcctgtaccatcgcacatggatgaaccaatcggcgaggaagacgacgagagcgaggacgaagacgaggatgaagatggaccagacgagtcgggggattcagacgagggcgaggatgaagacgcaaaagaagagtcgcctcactaaaaaagtaagttttctctatctcattgcttctttggttgctcggataatgcttagctgttgttggtaaactcccagtagccaacgctcatcatttgcaatttttattttcgcatatatcagttgaccacgacaagtttgccaagatgcgtgcggaacactacaagatgaaggaggcgcttcaattggggcatgagctggcagaggaagagctgagtgcgctggacagtcctgatccaaacgatatgccagtgccgccattaccgtcgtttgctcaacagtcgaacgcggctaggctgtcacgggaggctggatcgaacaagctgaaggaggaccttgaaaacatggagctttagaggtttggagttggctttgaccatggctatggctacgtattctgaacgacataaaggacgctcatttttcgctgcaggacattttttgagttgcagcacagaggggcaaggcggtgctctggactgctttatcgggctgctacgcgtgcgatttgtttacgttttttccggtttgttggccagcagtatttgtaggccctgcagctgggggtgggttgatcctctttctcttctcttctcttttctctttttccctcttctgatgtgtctcccaccccacaaccttctcctctgcccccagccgcatcggtcccaccgccgcaacccatcagcacaccatggccatcaaggaataccagcgcgagttcattgagtttgccatcaagaacgaggtcttgaagttcggagagttcaccctcaagtccggccgtatctcgccctacttcttgaacgcgggccttttcaacactggcgcctcgctctccaagatcggaaagttctacgccgctgccgtcagcgattcgggcattgagcacgacatcatctttgg

SEQ ID NO: 4 - DNA, 1362 bp, Mortierella formosensis
ccctctggcctgcaccaccgtcattgccttggccgaggccccctacaacaaggacacgccctactgcttcaaccgcaaggagaagaaggaccatggtgagggcggcacgattgttggatcagcactgaagggcaaggtcctggtcattgacgatgttatcaccgccggcaccgccatccgcgagtctgtccagatcattgaggactgcaaggcccaattggctggtgttttggtcgcggtggatcgtcaggagactggcaagaacggcgacatgtctgctatccaggaggtcgagagagactttggtgtccctgtcaaggccattgtgaccatgacccacatcatgcagtacatggaggagaagggaacctatggcgagcacttgacccagatgcgcgcttaccgggagaagtacggtgtttagagcaagcgaactctggatgggatgaagctcggtttcaatgcggcgagcgagggctctgttggatttttctcgtaatgcggggagacggacgcccggggaacgatgtgctcctgatcagtggttttcgagtgttctcgggacagcccgtcttgggaaaccaccgaacgatggctattaataataaatacccatacaacaacttttcctcagtgtggtagttggggtgtgatatcgccgtgcatgtccaaggcttcagctgcgcctggcgacgagatggaaggtcgtgggaaagaggcgtcgaaactgagctgtcaagaagaaagtaaaaaaaccgtcgtaaaatagagctgtgtcgtcaaatggcgtgtatggggtattcggcgcgacaggctatttgattccgatggggctccagacaaggcgccaggagctcatccaagtcgatcgcccgctgtacgacgcctctgtagtatggggacatgattctgctggtggatgtttctgcagccaccagaaaattgaagctcagcctgtaaaaaaaaatattacattgtggcgagcgagtcacttctctgttctccttttcatttccacccaccctcattccacatccattcaccaccgctcattcgcttcacaatggcagagactcttactcaccctcttgtccaggacggctggttcaaggagaccggcaccctctggcccggccaggccatgactctcgaggtcaaggagattctgcacgttgaaaagtcgctcttccaggacgtgctcgtcttccagtccacctcctacggcaacgtcctcgtcctcgacggcgtcatccaggccaccgagcgcgatgagttctcgtaagtgcgctagtgtgctagtgtgtgctcgagctcctcacctggagtcctctacctgatcattccatatctgtgattcgcatg

SEQ ID NO: 5 - DNA, 36 bp, Artificial Sequence
  The sequence is synthesized
gaccggaattccgacgctgacattacacatttatcc

SEQ ID NO: 6 - DNA, 47 bp, Artificial Sequence
  The sequence is synthesized
tgacggtggtgcaggccagagggccaaagatgatgtcgtgctcaatg

SEQ ID NO: 7 - DNA, 47 bp, Artificial Sequence
  The sequence is synthesized
ttgagcacgacatcatctttggccctctggcctgcaccaccgtcatt

SEQ ID NO: 8 - DNA, 31 bp, Artificial Sequence
  The sequence is synthesized
tgcggggtacccatgcgaatcacagatatgg

SEQ ID NO: 9 - DNA, 35 bp, Artificial Sequence
  The sequence is synthesized
tttcgctagcacgacgttgtaaaacgacggccagt

SEQ ID NO: 10 - DNA, 34 bp, Artificial Sequence
  The sequence is synthesized
aacaacaattggggctccaccgcggtggcggccg

SEQ ID NO: 11 - DNA, 25 bp, Artificial Sequence
  The sequence is synthesized
atgaccatcaaggattaccagcgcg

SEQ ID NO: 12 - DNA, 25 bp, Artificial Sequence
  The sequence is synthesized
atccttaaacaccgtacttctcgcg

(19) United States
(12) Patent Application Publication
(10) Pub. No.: US 2018/0237789 A1
(43) Pub. Date: Aug. 23, 2018

(54) MORTIERELLA ALPINE URACIL AUXOTROPH WITH URA5 GENE KNOCKED OUT THROUGH HOMOLOGOUS RECOMBINATION

(71) Applicant: JIANGNAN UNIVERSITY, Wuxi (CN)

(72) Inventors: Haiqin CHEN, Wuxi (CN); Wei CHEN, Wuxi (CN); Yongquan CHEN, Wuxi (CN); Guangfei HAO, Wuxi (CN); Xin TANG, Wuxi (CN); Zhennan GU, Wuxi (CN); Jianxin ZHAO, Wuxi (CN); Hao ZHANG, Wuxi (CN)

(73) Assignee: JIANGNAN UNIVERSITY, Wuxi (CN)

(21) Appl. No.: 15/954,805

(22) Filed: Apr. 17, 2018

Related U.S. Application Data
(62) Division of application No. 14/910,675, filed on Apr. 16, 2016, now Pat. No. 9,982,269.

(51) Int. Cl. C12N 15/80; C12N 9/10

(52) U.S. Cl. CPC C12N 15/80; C12N 9/1077; C12Y 204/0201

(57) ABSTRACT

It relates to a Mortierella alpine ATCC32222 uracil auxotroph strain and a construction method thereof. In the present invention, Mortierella alpine ATCC32222 is used as a material and undergoes gene knockout through an Agrobacterium tumefaciens mediated genetic manipulation technology, to obtain the Mortierella alpine uracil auxotroph. The method is of great significance for the basic theoretic researches of the oil producing fungus Mortierella alpine ATCC32222 and product development.

This application is the divisional application of U.S. Ser. No. 14/910,675 that claims priority to U.S. national phase of International Application No. PCT/CN2014/072350 Filed on 21 Feb. 2014 which designated the U.S. and claims priority to Chinese Application Nos. CN201310347934.8 filed on 9 Aug. 2013, the entire contents of each of which are hereby incorporated by reference.

### FIELD OF THE INVENTION

The present invention relates to a Mortierella alpina uracil auxotrophic strain and its construction method. It is in the field of biotechnology engineering.

### BACKGROUND OF THE INVENTION

Mortierella alpina is an important arachidonic acid (ARA) industrial production fungus. The produced polyunsaturated fatty acids (PUFAs) have a reasonable composition that contains high level of ARA, which have a record of complete safe for applications in food. By far, the studies on M. alpina were mainly focused on the strain breeding and the optimization of fermentation conditions. The gene transformation system of M. alpina has not been well established. This is a great obstacle to the studies on the mechanism of fatty acid synthesis and metabolic engineering of M. alpina. Auxotrophic marker, antibiotic resistance marker and fluorescent reporter gene are three well-used selective marker for gene transformation in filamentous fungi. The auxotrophic is applicable for industrial production, because there is no residual exogenous resistance gene. Therefore, the auxotrophic strains are important for industrial breeding microorganisms, genetics, medicine, food and biotechnology engineering. Currently, the auxotrophic strains of filamentous fungi are mainly generated by the mutation method, which is inefficient and often causes random unknown mutations in the genome DNA sequences. These unknown mutations may bring unpredictable problems for the future genetically engineering and industrial production.

Constructing auxotrophic through homologous recombination can knock out the target gene without affecting the function of the other genes. Compared to random mutations, homologous recombination is more efficient and repeatable. Therefore, directly interrupt the target gene via homologous recombination is an optional way in generating auxotrophic strains. In filamentous fungi, homologous recombination is affected by many factors: the length, similarity, G/C percentage, transcription of target gene, non-homologous end joining and chromatin structure, as well as the transformation method. In some yeast, homologous recombination could be achieved with a relative short homologous DNA sequence of 50 bp to 100 bp. Whereas in filamentous fungi, homologous DNA sequence often needs to be over 1K bp even longer. The homologous recombination probability may differ a lot among strains and genes, which may strongly affect the experiment. Orotate phosphoribosyltransferase (OPRTase) is a key enzyme during uracil metabolic in M. alpina. The M. alpina auxotroph could be generated by inactivation the OPRTase coding gene ura5. However, ura5 gene has an extremely important role in the cellular processes of life, resulting in very sensitive self-defense and repair mechanisms of the role of eukaryotic cells. Construction of ura5 uracil auxotrophic strain using gene knockout method in filamentous fungi is seldom publicly reported.

The gene manipulation system of filamentous fungus has not been well established, mainly because it is difficult to be transformed. Agrobacterium tumefaciens-mediated transformation (ATMT) method has been gradually applied in filamentous fungi, which have four advantages compared to other transformation methods. First, the recipient could be spores or mycelia without preparing protoplasts. Second, the mononuclear spores as a recipient can avoid transformants instability caused by multicore mycelium. Third, the method uses a natural transformation vector system having high conversion efficiency and high success rate. The plasmid can hold large fragments of DNA with a single copy insertion into genome. Fourth, a relative higher homologous recombination rate can be achieved.

The M. alpina uracil auxotrophic strain is the prerequisites of the gene manipulation of this important industrial PUFA production fungus. This uracil auxotrophic strain could be applied in both theoretical research of fatty acid synthesis and accumulation and genetically engineering to breeding super PUFA production industrial strain.

### SUMMARY OF THE INVENTION

The object of the present invention is to provide a uracil auxotrophic strain of M. alpina. The auxotroph was constructed by deletion of the 18 bp (213 bp to 230 bp) of the M. alpina ATCC 32222 ura5 gene (654 bp).

The sequence of the homologous DNA arms refers to the 1393 bp (from −1180 bp to +212 bp) up-stream and the 1362 bp (from +231 bp to +1592 bp) down-stream of the ura5 gene of M. alpina ATCC 3222 genome sequence (DDBJ/EMBL/GenBank accession ADAG00000000, first version ADAG01000000).

The present invention also provides a method of constructing the uracil auxotrophic strain of M. alpina comprising: obtaining the ura5 knockout DNA fragment; constructing the knockout plasmid pBIG4KOura5; transformation of A. tumefaciens using pBIG4KOura5; ATMT of M. alpina using A. tumefaciens that containing pBIG4KOura5; screening and identifying uracil auxotroph to obtain uracil auxotrophic strains. As illustrated in FIG. 1, the multiple cloning site (MCS) DNA fragment is PCR amplified from plasmid pBluescript II SK+. Digest the MCS fragment and plasmid pBIG2RHPH2 with NheI/MunI and EcoRI/XbaI, followed by the ligation to form plasmid pBIG4. Ligate the up- and down-stream knockout DNA arms with fusion PCR to form the knockout DNA fragment KOura5. Digest the KOura5 fragment and plasmid pBIG4, followed by the ligation to form plasmid pBIG4KOura5. Transform A. tumefaciens C58C1 using plasmid pBIG4KOura5. ATMT M. alpina disrupts the ura5 gene to construct uracil auxotrophic strain of M. alpina.

Specifically, this invention provides a M. alpina uracil auxotrophic strain, which is generated by inactivating the ura5 encoding orotate phosphoribosyltransferase (OPRTase).

According to one preferable embodiment of the present invention, the inactivation of the 654 bp ura5 gene is achieved by the deletion of the 18 bp (213 bp to 230 bp) DNA sequence.

The present invention also provides a method for the construction of M. alpina uracil auxotroph according to any of claim 1 and 2. Inactivate the M. alpina ura5 gene through deletion of the 18 bp (213 bp to 230 bp) DNA sequence by homologous recombination. The homologous DNA arms are the 1393 bp (from −1180 bp to +212 bp) up-stream and the 1362 bp (from +231 bp to +1592 bp) down-stream of the ura5 gene. The detailed steps are described as follows: obtaining the ura5 knockout DNA fragment; constructing the knockout plasmid pBIG4KOura5; transformation of A. tumefaciens using pBIG4KOura5; ATMT of M. alpina using A. tumefaciens C58C1-pBIG4KOura5 (CGMCC No. 7730); screening and identifying uracil auxotroph to obtain uracil auxotrophic strains.

In the present invention, the A. tumefaciens used is Agrobacterium tumefaciens C58C1, received from Professor Yasuyuki Kubo (Kyoto Prefectural University, Kyoto, Japan).

The starting A. tumefaciens plasmid is pBIG2RHPH2, received from Professor Yasuyuki Kubo (Kyoto Prefectural University, Kyoto, Japan), with sequence of SEQ No. 1.

According to a preferable embodiment of the present invention, the gene knockout plasmid is constructed as follows:

(a) amplifying the MCS DNA fragment is by PCR using plasmid pBluescript II SK+ as template;

(b) digesting MCS DNA fragment and plasmid pBIG2RHPH2 by EcoRI and XbaI, and ligating them together at the EcoRI and XbaI sites to form the plasmid pBIG4;

(c) PCR amplifying the up- and down-stream arms of ura5 gene and ligating them together by using fusion PCR to form knockout DNA sequence;

(d) digesting the KOura5 knockout DNA sequence and pBIG4 by EcoRI and KpnI, and ligating them together to form plasmid pBIG4KOura5.

Preferably, the knockout DNA sequence in step (c) is constructed as the following steps:

designing the primers according to the sequence data of NCBI:

P1:
GACCGGAATTCCGACGCTGACATTACACATTTATCC
P2:
TGACGGTGGTGCAGGCCAGAGGGCCAAAGATGATGTCGTGCTCAATG
P3:
TTGAGCACGACATCATCTTTGGCCCTCTGGCCTGCACCACCGTCATT
P4:
TGCGGGGTACCCATGCGAATCACAGATATGG

subsequently, PCR amplifying up- and down-stream DNA fragments by using P1/P2 and P3/P4 with M. alpina ATCC 32222 genome DNA as template, then performing fusion PCR by using P1/P4 with up- and down-stream DNA fragments as templates to amplify the KOura5 knockout DNA sequence.

More preferably, the primers below are designed according to the sequence of pBluescript II SK+:

MCSF:
TTTCGCTAGCACGACGTTGTAAAACGACGGCCAGT
MCSR:
AACAACAATTGGGGCTCCACCGCGGTGGCGGCCG

Then the MCS DNA fragment in step (a) is amplified by PCR using primer pair MCSF/MCSR with pBluescript II SK+ as template.

The said ATMT gene knockout consists in using A. tumefaciens to transform M. alpina, specified as follows: mixing equal volume of 100 μL of A. tumefaciens and M. alpina spores, and spreading on the cellophane membrane placed on the IM solid medium, after co-cultivation, selecting the uracil auxotrophic strains of M. alpina.

The ATMT method comprises:

(i) separating the A. tumefaciens harboring pBIG4KOura5 (preserved at the temperature of −80° C.) by stripping on the TEP solid plate (containing 100 μg/mL rifampicin and 100 μg/mL kanamycin) to obtain single clone by cultured at the temperature of 30° C. for 48 h.

(ii)(2) transferring a single clone to 20 mL YEP medium (containing 100 μg/mL rifampicin and 100 μg/mL kanamycin) and culturing at the temperature of 30° C. for 48 h with shaking at 200 rpm in the dark;

(iii) collecting A. tumefaciens by centrifuging at 4000×g for 5 min, after removing the suspension, suspending the pellet by 5 mL of IM medium, followed by a centrifugation at 4000×g for 5 min, and then removing the suspension, adding 2 mL of IM medium to suspend the bacterium;

(iv) adjusting the concentration of the bacterium suspension to OD600=0.9, followed by a dark cultivation at the temperature of 30° C. to OD600=1.5;

(v) collecting the M. alpina spores, counting the number, then adjusting the spore concentration to 10⁶/100 μL;

(vi) mixing equal volume of 100 μL of A. tumefaciens and spores, spreading on the cellophane membrane placed on the IM solid medium, then incubating at the temperature of 23° C. for 48 to 96 h in a dark incubator;

(vii) transferring the cellophane membrane onto GY plate containing 100 μg/mL cefotaxime and 100 μg/mL spectinomycin, then incubating at the temperature of 25° C. to 30° C. until spores appears.

In this invention, the IM solid medium is composed of 1.74 g/L K₂HPO₄, 1.37 g/L KH₂PO₄, 0.146 g/L NaCl, 0.49 g/L MgSO₄.7H₂O, 0.078 g/L CaCl₂, 0.0025 g/L FeSO₄.7H₂O, 0.53 g/L (NH₄)₂SO₄, 7.8 g/L MES, 1.8 g/L glucose, 0.5% glycerol and 20 g/L agar.

The present invention builds a M. alpina uracil auxotrophic strain using the ATMT gene knockout method, based on the bioinformatics analysis of M. alpina ATCC 32222 genome, after a lot of practice. The M. alpina uracil auxotrophic strain has genetic stability after several generations, and fatty acid composition shows no significant difference with the wild-type strain. This uracil auxotroph can be used as a recipient strain for genetic engineering.

The A. tumefaciens C58C1-pBIG4KOura5 obtained according to this invention was preserved in China General Microbiological Culture Collection Center (CGMCC) since Jun. 28, 2013, with the accession number CGMCC No. 7730. The address of CGMCC is the Institute of Microbiology, Chinese Academy of Sciences, No. 1, Beichen West Road, Chaoyang District, Beijing, China, Zip code 100101.

### DESCRIPTION OF THE ATTACHED DRAWINGS

FIG. 1 is the schematic diagram of the construction of the plasmid for gene knockout;

FIG. 2 is the analysis diagram of the conserved region of M. alpina OPRTase;

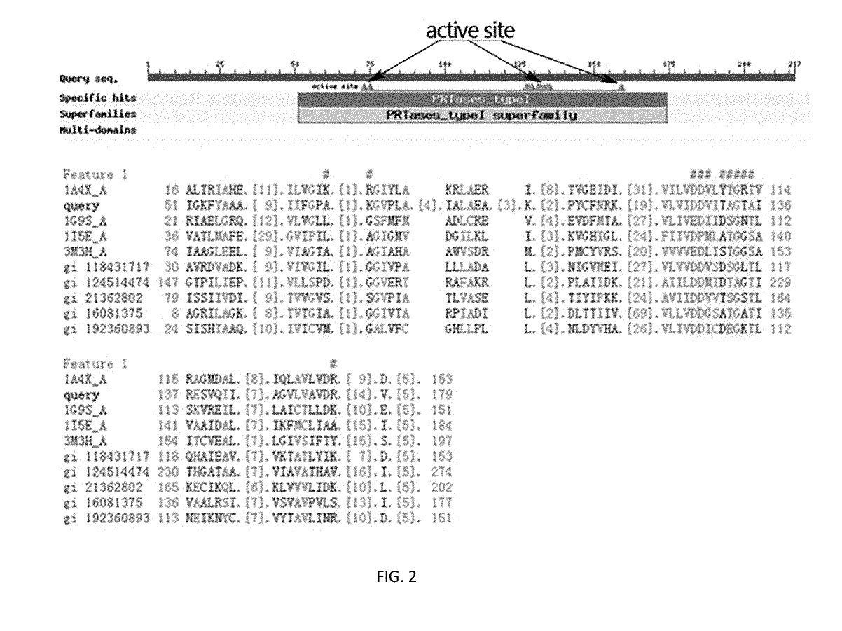

FIG. 3 is the agarose gel electrophoresis of the fusion PCR fragments.

### EMBODIMENTS

The following Embodiments further illustrate the present invention. The experimental methods without indicating specific conditions in the following examples will be performed generally in accordance with the manual of molecular cloning experiments.

### Example 1: The Bioinformatics Analysis of M. alpina Genome

Compare the protein coding sequence, which was predicted based on the M. alpina ATCC 32222 genome (DDBJ/EMBL/GenBank accession ADAG00000000, first version ADAG01000000), to the database NR (www.ncbi.nlm.nih.gov), KOGs and COGs, KEGG, Swiss-Prot, UniRef100, and BRENDA using BLAST (E-value 1E-5). Search InterProScan against protein domain databases with default parameter settings. Predict the 654 bp ura5 gene coding sequence and find no intron exists. Search the M. alpina genome sequence with the sequence of ura5 gene for the up- and down-stream sequence.

### Example 2: Obtaining the KOura5 DNA Fragment

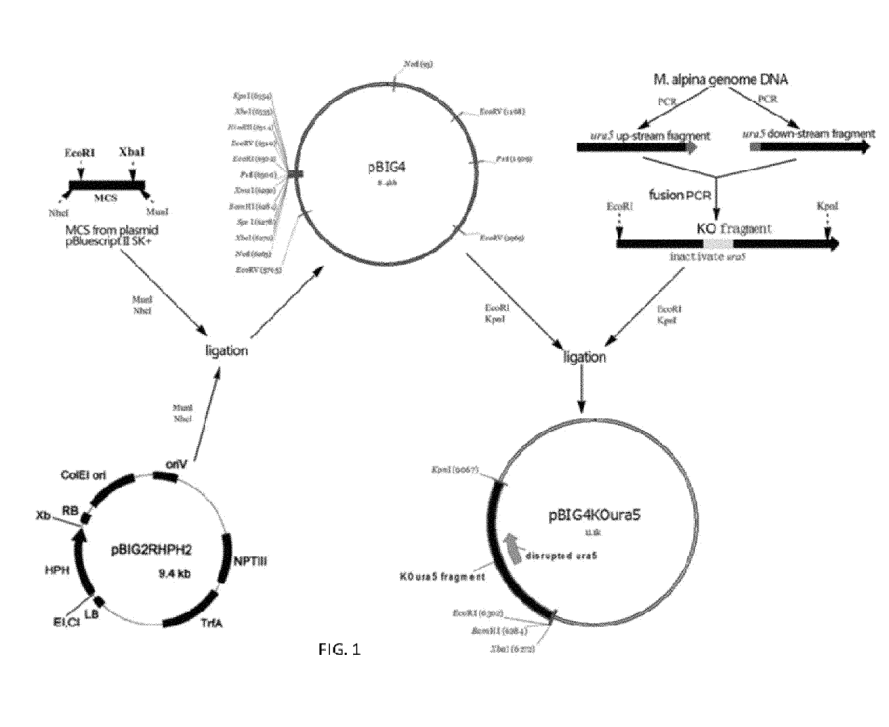

Find the conserved active site of the protein sequence of M. alpina OPRTase (FIG. 1). Design different homologous arms to disrupt ura5 gene. After many practice and comparison of the different plans, confirm that the effective homologous DNA arms are the 1393 bp (from −1180 bp to +212 bp) up-stream and the 1362 bp (from +231 bp to +1592 bp) down-stream of the ura5 gene. The details of the success experimental plan are as follows:

First, design primers based on the bioinformatics analysis.

P1:
GACCGGAATTCCGACGCTGACATTACACATTTATCC
P2:
TGACGGTGGTGCAGGCCAGAGGGCCAAAGATGATGTCGTGCTCAATG
P3:
TTGAGCACGACATCATCTTTGGCCCTCTGGCCTGCACCACCGTCATT
P4:
TGCGGGGTACCCATGCGAATCACAGATATGG

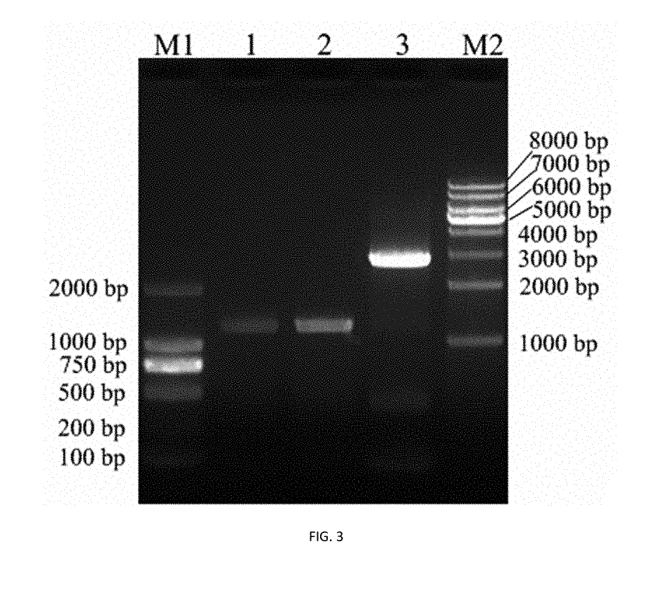

Introduce EcoRI and KpnI into the 5′ site of P1 and P4. PCR amplify the up- and down-stream fragments of ura5 gene with M. alpina genome as template, followed by a gel purification. Ligate the two fragments using fusion PCR with primer pair P1/P4 using the up- and down-stream fragments as templates. FIG. 3 is the results of the agarose gel. As shown in the picture, M1 is the D2000 Marker; channel 1 is the up-stream fragment; channel 2 is the down-stream fragment; channel 3 is the fusion PCR product; M2 is the 1 kb ladder Marker. Sub-clone the fragment of fusion PCR into the pEGMT-easy vector and analyze the sequence by ABI PRISM 3730.

### Example 3: Construction of the Knockout Plasmid pBIG4KOura5

Design primers according to the sequence of plasmid pBluescript II SK+:

MCS Forward:
TTTCGCTAGCACGACGTTGTAAAACGACGGCCAGT
MCS Reverse:
AACAACAATTGGGGCTCCACCGCGGTGGCGGCCG

MCS DNA fragment was amplified from plasmid pBluescript II SK+.

Digest the MCS fragment and plasmid pBIG2RHPH2 with EcoRI and XbaI, followed by a gel purification and ligation. The 10 μL ligation mixtures consisted of: MCS DNA fragment 2 μL, plasmid 2 μL, 10×T4 ligase buffer 1 μL, T4 ligase 1 μL and H₂O 4 μL. Ligate at the temperature of 4° C. for 12 h.

Directly transform the ligation product into Escherichia coli TOP10 competent cell. The electro transformation comprises:

(a) Take out 100 μL competent cells under sterile conditions, add 1 to 2 μL ligation product and mix.

(b) Transfer the mixture of step (a)(1) into cuvette, avoiding to make air bubbles.

(c) Transfer the cuvette into the Bio-Rad electroporation device, select the appropriate program and click pulse.

(d) Transfer the pulsed competent cell into 900 μL SOC medium and incubate at the temperature of 37° C. at 150 rpm for 1 h.

(e) Transfer 200 μL of the culture onto YEP plate (containing 100 μg/mL kanamycin) and spread with a sterile stick. Inverted incubate overnight at the temperature of 37° C.

Select the positive transformants and extract the plasmid. Analyze the sequence by ABI PRISM 3730. The resulted plasmid is named as pBIG4.

Digest KOura5 DNA fragment and plasmid pBIG4 with Nhe/MunI and EcoRI/KpnI, followed by the gel purification and ligation. Ligate with the ligase T4. Transform the reaction mixture into TOP10 competent, select positive clone and analysis of the DNA sequence proves ligation successful. The resulted plasmid is named as pBIG4KOura5.

The SOC medium was composed of 20 g/L Tryptone, 5 g/L yeast extract, 0.5 g/L NaCl, 2.5 mM KCl, 10 mM MgCl₂ and 20 mM glucose; The YEP solid medium was composed of 10 g/L Tryptone, 10 g/L yeast extract, 5 g/L NaCl and 20 g/L agar.

### Example 4: ATMT of M. alpina

The transformation was optimized according to the method referred to the open accessed articles, the detailed steps are as follows:

(i) Take out the A. tumefaciens C58C1 (harboring pBIG4KOura5) preserved at the temperature of −80° C. and separate by stripping on the TEP solid plate (containing 100 μg/mL rifampicin and 100 μg/mL kanamycin) to obtain single clone by cultured at the temperature of 30° C. for 48 h.

(ii) Transfer a single clone to 20 mL YEP medium (containing 100 μg/mL rifampicin and 100 μg/mL kanamycin) and cultured at the temperature of 30° C. for 48 h with shaking at 200 rpm in the dark.

(iii) Collect A. tumefaciens by centrifuging at 4000×g for 5 min. After remove the suspension, suspend pellet by 5 mL of IM medium, followed by a centrifugation at 4000×g for 5 min. After remove the suspension, add 2 mL of IM medium to suspend the bacterium.

(iv) Adjust the concentration of the bacterium suspension to OD600=0.9, followed by a dark cultivation at the temperature of 30° C. to OD600=1.5;

(v) Collect the M. alpina spores and count the number, then adjust the spore concentration to 10⁶/100 μL;

(vi) Mix equal volume of 100 μL of A. tumefaciens and spores and spread on the cellophane membrane placed on the IM solid medium, then incubate at the temperature of 23° C. for 48 to 96 h in a dark incubator;

(vii) Transfer the cellophane membrane onto GY plate containing 100 μg/mL cefotaxime, 100 μg/mL spectinomycin and 0.05 g/L uracil, then incubate at the temperature of 25° C. to 30° C. until spores appears.

Wherein, the liquid YEP medium was composed of 10 g/L Tryptone, 10 g/L yeast extract and 5 g/L NaCl.

### Example 5: Screening and Identification of M. alpina Uracil Auxotroph

(a) Scour the surface of the co-cultured template with 3 mL of saline solution. Collect the solution with 1.5 mL tube and filter with 25 μm membrane.

(b) Spread 200 μL of the solution onto GY plate (containing 1 mg/mL 5-FOA, 100 μg/mL spectinomycin, 100 μg/mL cefotaxime and 0.05 g/L uracil).

(c) Incubate the plate at the temperature of 25° C. for 5 to 10 days in the dark.

(d) Transfer the visible mycelium onto GY plate (containing 1 mg/mL 5-FOA, 100 μg/mL spectinomycin, 100 μg/mL cefotaxime and 0.05 g/L uracil), and cultivate at the temperature of 25° C. for 2 to 4 days in a dark incubator.

(e) Transfer the well grown mycelium in step (d) separately onto the SC plate containing uracil and the SC plate without uracil. Cultivate at the temperature of 25° C. for 2 to 4 days.

(f) Observe the growth of the mycelium on the two SC plates. Select the mycelium only grown on the SC plate containing uracil and then transfer them onto the GY medium slant containing 0.5 mg/mL 5-FOA.

(g) Culture the M. alpina spores of step (f) for 3 generations on GY medium slant containing 0.5 mg/mL 5-FOA. Repeat the experiment described in step (e) each generation.

(h) Identify the genetic stable strains as uracil auxotrophic phenotype and preserve on GY medium slant containing 0.5 mg/mL 5-FOA.

(i) Extract the genome of the uracil auxotroph and PCR for ura5 gene with the primers below:

Forward:
ATGACCATCAAGGATTACCAGCGCG
Reverse:
ATCCTTAAACACCGTACTTCTCGCG

Purify the PCR product and analyze sequence by ABI PRISM 3730. Identify the gene as loss of 213 bp to 230 bp.

### Example 6: Extraction and Analysis of the Fatty Acids of M. alpina Uracil Auxotroph

(a) Culture the M. alpina prototrophic strain and three M. alpina uracil auxotroph strains screened in Example 5 in ferment medium (adding extra 0.05 g/L uracil for auxotroph strains) at the temperature of 25° C. at 200 rpm for 7 to 14 days.

Wherein, the ferment medium is available on the market, and is composed of 50 g/L glucose, 2.0 g/L L-Ammonium tartrate, 7.0 g/L KH₂PO₄, 2.0 g/L Na₂HPO₄, 1.5 g/L MgSO₄.7H₂O, 1.5 g/L Yeast extract, 0.1 g/L CaCl₂.2H₂O, 8 mg/L FeCl₃.6H₂O, 1 mg/L ZnSO₄.7H₂O, 0.1 mg/L CuSO₄.5H₂O, 0.1 mg/L Co(NO₃)₂.6H₂O and 0.1 mg/L MnSO₄.5H₂O.

(b) Collect mycelia and freeze-dry.

(c) Mix 100 mg mycelia (dry weight) with 2 mL of 4 mol/L HCl.

(d) Water bath at 80° C. for 0.5 h, then at −80° C. for 15 min. Repeat once. Then water bath at 80° C. for 0.5 h.

(e) Cool down the mixture to room temperature, add 1 mL methanol and well mix.

(f) Add 1 mL chloroform and shake for 10 min, followed by centrifuge at 6000×g for 3 min. Collect the chloroform.

(g) Repeat step (f) for two times.

(h) Combine chloroform (3 mL), add 1 mL saturated NaCl solution, mix well and centrifuge at 3000×g for 3 min. Transfer the chloroform into a new tube. Add 1 mL chloroform in the residual liquid, followed by centrifugation at 3000×g for 3 min. Combine all the chloroform (4 mL)

(i) After drying by nitrogen blow, add 1 mL ethyl ether. Transfer the solution to a clean and weighed tube, followed by drying by nitrogen blow, then weigh it to obtain total fatty acid weight. The total fatty acid content of prototrophic and three uracil auxotroph M. alpina are listed in Table 1.

TABLE 1
The total fatty acid of prototrophic and
three uracil auxotroph M. alpina

| | Dry Weight | Fatty Acid Content |
|---|---|---|
| Strains | (mg) | (%) |
| MA | 46.2 | 30.64 ± 0.035 |
| MAU1 | 49.0 | 30.56 ± 0.026 |
| MAU2 | 50.5 | 30.72 ± 0.036 |
| MAU3 | 52.1 | 30.60 ± 0.029 |

(j) Analyze the fatty acids by GC

The total fatty acid composition of prototrophic and three uracil auxotroph M. alpina are listed in Table 2.

TABLE 2
The total fatty acid composition of prototrophic and
three uracil auxotroph M. alpina

| | Fatty Acid Composition (%) | | | | | | | | |
|---|---|---|---|---|---|---|---|---|---|
| Strains | 16:0 | 18:0 | 18:1 | 18:2 | 18:3 | 20:3 | 20:4 | 22:0 | 24:0 |
| MA | 14.98 | 10.73 | 8.91 | 15.60 | 2.61 | 1.97 | 34.53 | 1.27 | 1.79 |
| MAU1 | 13.59 | 10.98 | 9.40 | 17.17 | 2.59 | 1.81 | 34.50 | 1.21 | 1.57 |
| MAU2 | 14.4 | 11.35 | 9.67 | 16.83 | 2.56 | 1.90 | 34.84 | 1.26 | 1.62 |
| MAU3 | 13.56 | 10.48 | 9.17 | 16.43 | 2.43 | 1.66 | 34.16 | 1.20 | 1.54 |

The results of experiments show that the uracil auxotrophic M. alpina that constructed according to the method of the experiments has genetic stability after cultured for multiple generations, and its fatty acid analysis shows no distinguished difference between that of prototrophic M. alpina strains. The strain constructed according to the method of the present invention could be taken as the recipient strain for genetic engineering.

Above-mentioned preferred embodiments are not intended to limit the present invention. Those skilled in the art, without departing from the spirit and scope of the present invention, can make a variety of variations and modifications. Therefore, the protection scope of the present invention shall be based on the claims.

What is claimed is:

1. A method of constructing a Mortierella alpina ATCC 32222 uracil auxotroph strain, which is generated by inactivating the ura5 encoding orotate phosphoribosyltransferase (OPRTase), in which the inactivation is achieved through the deletion of the 18 bp (from 213 bp to 230 bp) of the 654 bp ura5 genome DNA having a nuclei acid sequence shown as SEQ ID NO: 2, characterized in that it inactivates ura5 gene through the deletion of the 18 bp (from 213 bp to 230 bp) of the 654 bp in Mortierella alpina by homologous recombination, in which the homologous DNA sequences are the 1393 bp (from −1180 bp to +212 bp) up-stream and the 1362 bp (from +231 bp to +1592 bp) down-stream of the M. alpina ura5 genome DNA sequence having a nuclei acid sequence shown as SEQ ID NO: 3, the steps of the said method are as follows: acquisition of the up- and down-stream sequences of ura5 gene; construction of knockout plasmid pBIG4KOura5; transformation of Agrobacterium tumefaciens C58C1 with plasmid pBIG4KOura5; transformation of M. alpina with the A. tumefaciens C58C1 (harboring pBIG4KOura5) using the Agrobacterium tumefaciens-mediate transformation (ATMT) method, then screening and identifying the uracil auxotroph to obtain the uracil auxotrophic stain of M. alpine; wherein the uracil auxotrophic stain of M. alpine is Mortierella alpina MAU1 deposited at the General Microbiology Culture Collection Center of China Committee for Culture Collection of Microorganisms under accession number CGMCC No. 8414.

2. The method according to claim 1, characterized in that the starting plasmid of Agrobacterium tumefaciens used for gene knockout is pBIG2RHPH2 having a nuclei acid sequence shown as SEQ ID NO: 1.

3. The method according to claim 2, characterized in that construction of the gene knockout plasmid comprises:
(a) amplifying MCS DNA fragment by PCR using plasmid pBluescript II SK+ as template;
(b) digesting MCS DNA fragment and plasmid pBIG2RHPH2 by EcoRI and XbaI, and ligating them together at the EcoRI and XbaI sites to form the plasmid pBIG4;
(c) PCR amplifying the up- and down-stream arms of ura5 gene and ligating them together by using fusion PCR to form knockout DNA sequence;
(d) digesting the KOura5 knockout DNA sequence and pBIG4 by EcoRI and KpnI, and ligating them together to form plasmid pBIG4KOura5.

4. The method according to claim 3, characterized in that the knockout DNA sequence in step (c) is constructed as the following steps:
designing the primers according to the sequence data of NCBI:
P1:
(SEQ ID NO: 5)
GACCGGAATTCCGACGCTGACATTACACATTTATCC
P2:
(SEQ ID NO: 6)
TGACGGTGGTGCAGGCCAGAGGGCCAAAGATGATGTCGTGCTCAATG
P3:
(SEQ ID NO: 7)
TTGAGCACGACATCATCTTTGGCCCTCTGGCCTGCACCACCGTCATT
P4:
(SEQ ID NO: 8)
TGCGGGGTACCCATGCGAATCACAGATATGG
subsequently, PCR amplifying up- and down-stream DNA fragments by using P1/P2 and P3/P4 with M. alpina ATCC 32222 genome DNA as template, then performing fusion PCR by using P1/P4 with up- and down-stream DNA fragments as templates to amplify the KOura5 knockout DNA sequence.

5. The method according to claim 4, characterized in that the following primers are designed according to the sequence of pBluescript II SK+:
MCSF:
(SEQ ID NO: 9)
TTTCGCTAGCACGACGTTGTAAAACGACGGCCAGT
MCSR:
(SEQ ID NO: 10)
AACAACAATTGGGGCTCCACCGCGGTGGCGGCCG
then the MCS DNA fragment in step (a) is amplified by PCR using primer pair MCSF/MCSR with pBluescript II SK⁺ as template.

6. The method according to claim 5, characterized in that the A. tumefaciens mediated gene knockout method consists in using the ATMT method to transform M. alpina, specified as follows: mixing equal volume of A. tumefaciens and M. alpina spores, then spreading on the cellophane membrane placed on the IM solid medium, after co-cultivation, screening and obtaining the uracil auxotrophic strains of M. alpina.
